(19) United States
(12) Patent Application Publication
(10) Pub. No.: US 2020/0229925 A1
(43) Pub. Date: Jul. 23, 2020

(54) ADJUSTABLE PERCUTANEOUS ANNULOPLASTY DEVICES, DELIVERY SYSTEMS, A METHOD FOR PERCUTANEOUSLY DEPLOYING AN ANNULOPLASTY DEVICEA AND A METHOD PERFORMED BY ONE OR MORE PROCESSING DEVICES

(71) Applicant: Kephalios S.A.S., Paris (FR)

(72) Inventors: Piergiorgio TOZZI, Lausanne (CH); Daniel HAYOZ, Villars-sur-Glâne (CH); Marco GARD, Borgomasino (IT); Daniele ZANOTTI, Aix en Provence (FR)

(21) Appl. No.: 16/632,178

(22) PCT Filed: Jul. 18, 2017
(86) PCT No.: PCT/EP2017/068164

§ 371 Date: Jan. 17, 2020

(51) Int. Cl. A61F 2/24

(52) U.S. Cl. CPC A61F 2/2448; A61F 2210/0014; A61F 2250/001; A61F 2/2466

(57) ABSTRACT

An adjustable annuloplasty device (1) which comprises a tubular, longitudinally extending cage (2) having a basically annular shape or being adapted to be brought into an annular shape upon release from a delivery device (3). The annuloplasty device additionally comprises a separate, tubular, longitudinally extending base (4) having at least a part of the basically annular shape of the cage (2) or being adapted to be brought into at least partially into the annular shape of the cage (2) upon release from a delivery device. The tubular cage (2) is attachable to the base (4) and the base (4) is flexible such that the annular shape of the base (4) is adjustable.

The present invention relates to adjustable annuloplasty devices, delivery systems, a method for deploying an annuloplasty device and a method performed by one or more processing devices. Some non-limiting examples focus on treating atrioventricular cardiac valves such as the mitral valve or the tricuspid valve, but the concept, function and benefit are not limited to these valves.

The performance of native valves, such as the mitral valve, may be reduced due to functional regurgitation. In functional regurgitation the ventricle (typically the left ventricle) is distorted or dilated. As a result the papillary muscles that support the leaflets of the native valve are stretched and the valve leaflets can no longer coapt properly.

Further, the performance of native valve may be reduced to a degenerative valve disease. Degenerative valve disease refers to a spectrum of conditions, in which morphologic changes in the connective tissue of the mitral valve cause structural lesions that prevent normal function of the mitral apparatus. One example of degenerative is annular dilation in the cross-section of the native valve.

Annuloplasty (e.g. mitral or tricuspid annuloplasty) is the implantation of an annuloplasty device (e.g., mitral ring or tricuspid ring) to deform and/or reinforce the valve annulus to correct insufficient valve function which may be caused by functional and/or degenerative regurgitation. During a classical annuloplasty procedure, the surgeon gauges the valve annulus and chooses a fixed size annuloplasty device accordingly. This procedure is performed in an open heart surgery on the arrested heart under cardiopulmonary bypass. However, open heart surgery and cardiopulmonary bypass impose significant risks on the patient. In particular, fragile patients have a high mortality rate in such surgeries. Furthermore open heart surgery requires prolonged hospital stays and additional care.

A further limitation of the classical procedure or known transcatheter annuloplasty devices is that, after implantation of an annuloplasty device, the size and geometry of the heart and the treated valve annulus may vary over time. For example, a dilated heart annulus may further widen or as in functional regurgitation the papillary muscles are further dilated. A fixed-size annuloplasty device may as a result become ineffective over time or inappropriate for the size of the valve annulus. This causes recurrent mitral regurgitation and poor clinical outcome. The common practice is to implant undersized rings in order to overcome the risk of recurrent regurgitation. Too small rings however, may result in hypertension which in turn leads to a further dilatation stress of the annulus. Furthermore, a small orifice area in the mitral valve may cause mitral stenosis.

WO 2016/174669 A1, filed by Valtech Cardio Ltd., discloses an annuloplasty structure having a primary body portion and a contraction member extending along a contracting portion of the annuloplasty structure. Further the structure comprises an actuatable adjustment mechanism coupled to the contraction member, that when actuated, adjusts a length of the annuloplasty structure by applying tension to the contraction member. The adjustment mechanism also includes a contraction-member-protecting element, having a first end coupled to the primary body portion of the annuloplasty structure, and a second end coupled to the adjustment mechanism. The contraction member extends from the adjustment mechanism via the contraction-member-protecting element to the primary body portion of the annuloplasty structure.

US 2016/0331534 A1, filed by Valcare, discloses a repair of heart valves through percutaneous trans-catheter delivery and fixation of annuloplasty rings to heart valves via a transapical approach to accessing the heart. A guiding sheath may be introduced into a ventricle of the heart through an access site at an apex of the heart. A distal end of the guiding sheath can be positioned retrograde through the target valve. An annuloplasty ring arranged in a compressed delivery geometry is advanced through the guiding sheath and into a distal portion of the guiding sheath positioned within the atrium of the heart. The distal end of the guiding sheath is retracted, thereby exposing the annuloplasty ring. The annuloplasty ring may be expanded from the delivery geometry to an operable geometry. Anchors on the annuloplasty ring may be deployed to press into and engage tissue of the annulus of the target valve.

WO 2015/121075 discloses an annuloplasty device which is adjustable. Three portions of an outer wall are more rigid than opposite portions of an inner wall. The inner wall is adapted to be displaced inwardly by an actuation element while the outer wall remains basically constant.

The problem of the invention is to provide a percutaneously implantable annuloplasty device, which allows an adjustment, i.e. fitting, of the annuloplasty device to the native valve annulus. In particular the problem of the invention is to increase coaptation between valve leaflets.

According to the invention the problem is solved with different aspects of the invention according to the independent claims and their characterizing features.

A first aspect of the invention concerns an adjustable percutaneous annuloplasty device, which comprises a longitudinally extending cage. The cage has a basically annular shape or is adapted to be brought into an annular shape upon release from a delivery device. The annuloplasty device additionally comprises a separate, longitudinally extending base having at least a part of the basically annular shape of the cage or being adapted to be brought into at least partially into the annular shape of the cage upon release from a delivery device. The cage is attachable to the base and the base comprises at least one flexible element such that the annular shape of the base is adjustable. The flexible element allows an expansion and/or compression along a longitudinal direction of the base. The expansion and/or compression allow a reliable adjustment during the implantation process before a final anchoring of the device.

Thereby, an annulus of a valve may be shaped such that a coaptation of leaflets of a heart valve is increased. The effect of the adjustment is a targeted reduction of the distances between opposite portion of the annulus, with the consequence of increase coaptation of the leaflets.

The proposed device has the advantage of having a two-part form. Thereby, a base can be introduced and adjusted first and an optimal reshaping of the annulus can be achieved. The separate cage allows a reinforcement of the base. The two part form allows a simplification of the device and thus simplifies handling the device and simplifies a production.

The shape of the cage and the elongate base allow an insertion of both components of the annuloplasty device into a sleeve of a delivery device and thus a percutaneous implantation.

The cage may be tubular. As used herein the term “tube” or “tubular” is intended to cover closed or partly open cross-sections, which means that the tube may have a cross section in a closed form generally in the form of an “O” or a “D”, or a partly open form generally in the form of a “C” or an elongated “C”. In preferred embodiments, the cage may have a closed cross-section in the form of an “O” or a “D”.

A basically “annular shape” as used herein is intended to cover any shape for circumscribing at least a majority of a periphery of a valve annulus. An annular shape may be closed (e.g. generally “O” shaped or generally “D” shaped) or an annular shape may be open (e.g. generally “C” shaped). An annular shape does cover non round geometries (e.g. “D” shapes, elongated “C” shapes”) as well as round geometries (e.g. generally “O” shaped or generally “C” shaped). An annular shape may be in a non-planar 3D shape, e.g. generally a saddle shape. The annular shape may be three dimensionally bent “O”, “C” or “D” shaped.

As utilized herein the terms “distal” and “proximal” are used in relation to the annuloplasty device when held in a delivery device and a user operating this delivery device.

In a preferred embodiment, the cage and the base are independently insertable into an implantation site. The cage and the base may be adapted to be connected to each other at the implantation site. Thereby, a design of both devices may be simplified in comparison to a single fully integrated device.

In a preferred embodiment, the base includes at least one flexible element and at least one rigid element, wherein the flexible element allows an expansion and/or compression along a longitudinal direction of the base. The expansion and/or compression allow a reliable adjustment during the implantation process before a final anchoring of the device.

In a preferred embodiment, the base comprises at least two rigid elements which are connected by a flexible element.

In a preferred embodiment, the base comprises alternating rigid and flexible elements. Thereby, the base includes an accordion like structure which allows a longitudinal expansion and/or compression, wherein each flexible element may be shaped independently such as to bring the native valve in a desired shape.

In a preferred embodiment, the at least one rigid element is arranged movably within the base such that it is pushable towards an inner annular area. Thereby, the native valve may additionally be adjusted by moving a section, i.e. a rigid element, towards the inner annular area. As a result, the shape of the annular valve may be adjusted in greater detail.

In a preferred embodiment, the at least one rigid element includes an interface, preferably an opening, for a connection to the cage. Thereby, the base and the cage may be fixedly connected to each other. In particular, the cage may comprise anchors, which may extend through the opening.

In a preferred embodiment, the cage comprises an outer and an inner wall. At least one portion of the outer wall is more rigid than opposite portion(s) of the inner wall. The inner wall is arranged nearer to an inside area defined by the annular shape than the outer wall. The inner wall is adapted to be displaced inwardly at least along less rigid portion(s) of the circumference upon actuation by at least one actuation element while the outer wall remains basically constant. As a consequence of the difference in rigidity, the inner wall is at least along a part of the length displaceable inwardly upon actuation by an actuation element while the outer wall remains constant, which leads to a reshaping of the valve.

In a preferred embodiment, the outer wall is more rigid along its entire length than the at least a part of the, preferably the entire, length of the inner wall. Thereby, the natural valve may be adjusted from all sides of the annulus. In this design the adjustment can be done anywhere along the annulus perimeter, allowing an increased freedom to the surgeon to improve coaptation locally only where needed, at the minimum expense of valve orifice reduction

In a preferred embodiment, the base and/or cage and/or commissural anchor(s) (9, 20) is/are made from or comprise a shape memory alloy, preferably nitinol. Thereby, the base and or cage may adapted in situ during or after the implantation, e.g. for anchoring the base and/or cage. The base and/or cage may also be made from or include a ferromagnetic shape memory alloy.

Cage and base may also be made from combinations of shape memory materials and non-shape memory materials such as metals like stainless steel, cobalt-chrome or titanium.

The outer wall of the cage may also be made from a shape memory alloy and the inner wall may be from another metal alloy which is plastically deformable by a balloon catheter.

In a preferred embodiment, the cage is made from or comprises a shape memory alloy such that the cage comprises a deployed and an undeployed state and such that in the deployed state the cage is adapted to assume the basically annular shape or being adapted to be brought into an annular shape upon release from a delivery device. Thereby, the cage assumes the annular shape without further intervention by a user and a simple handling during implantation, in particular while releasing the cage from a delivery device, is facilitated. Furthermore, the cage may reinforce an annular shape of the base.

In a preferred embodiment, the base is made from or comprises a shape memory alloy such that the cage comprises a deployed and an undeployed state and such that in the deployed state the base is adapted to assume the basically annular shape or being adapted to be brought into an annular shape upon release from a delivery device. Thereby, the base assumes the annular shape without further intervention by a user and a simple handling during implantation, in particular while releasing the cage from a delivery device, is facilitated.

In a preferred embodiment, the cage comprises anchors for engaging an annulus and/or for connecting the cage to the annular base. Thereby, the cage may be fixedly attached to the native valve annulus and/or base. Additionally or alternatively, the base comprises anchors for engaging an annulus and/or for connecting the base to the annular base.

In a preferred embodiment, the anchors are made from or comprise a shape memory alloy. Thereby the anchors are adapted to engage a tissue and/or the base and/or the cage. In particular, the anchors are adapted to move outwardly upon release from a delivery device. Thereby, the anchors are deployable by retracting a sheath.

Alternatively or additionally, the anchors are plastically deformable. This does not exclude that the anchors include a shape memory alloy. The anchors may include a plastically deformable part and a shape memory alloy. The delivery device may include a retractable or pushable mandrel to push the anchors out from an inside.

In a further preferred embodiment, the anchors may be deployable by rotation of the cage or one of the sleeves. In one embodiment, the inner sleeve comprises slits. Alternatively or additionally at least one of sleeves may comprise a window. The inner sleeve may rotatable. The inner sleeve is rotated until the slits align with the anchors which are the deployed, preferably with their shape memory properties.

In a preferred embodiment, the anchors comprise pins with a longitudinal body part and a preferably sharp tip. Thereby, the anchors may pierce through tissue of the native valve annulus and/or penetrate a part of the base. The pins may be bent. The anchors are preferably bioerodible.

In a preferred embodiment, the at least one rigid element includes an opening, wherein the anchors extend through or are adapted to extend through said opening. Thereby, the cage and the base are rigidly attachable to each other.

In a preferred embodiment, the annuloplasty device additionally comprises a transmission line, preferably a catheter, for adjusting the cage and/or annulus base, wherein the transmission line preferably comprises a lumen for a manipulator for adjusting the annular shape of the annuloplasty device. The manipulator may be balloon catheter. The transmission line is particularly preferred a tube. Thereby, a shape of the device may be adjusted directly after implantation as well as later, i.e. months or years after the implantation, in order to adapt the device to an altered native annulus. In a preferred embodiment, the transmission line is connected to a proximal end of the base and/or cage. The manipulator may push an annularly inner part towards an annular center of the device. Thereby, the annulus is deformed such that leaflets of a native valve are brought closer together and a coaptation between the leaflets is improved.

In a preferred embodiment, the base and/or cage comprise an undeployed substantially linear shape and a deployed annular shape. Thereby, a trans-catheter approach with smaller cross-sections is enabled. Other undeployed shapes than a straight linear shape, like a C-shape are possible. The tube might for example be delivered in a generally linear undeployed state and annular in a deployed state.

In a preferred embodiment, a distal end of the cage and/or base is spaced apart from a proximal section of the cage and/or base. Thereby, the annular shape is open, which allows the annuloplasty device to be shaped to a large native annulus. As a result, the device can be personalised to a range of naturally occurring valve sizes and fewer device sizes, i.e. only one size, need to be produced and stored.

Another aspect of the invention concerns another adjustable percutaneous annuloplasty device. The adjustable annuloplasty device comprises an elongate base, which has a basically annular shape. The base has a deployed state and an undeployed state. The base is flexible such that the annular shape of the base is adjustable during and/or after implantation. The base is made from or comprises a shape memory alloy such that in the deployed state upon release from a delivery device the base has an annular shape, preferably a D-shape. Thereby, a stable annuloplasty device is provided.

In a preferred embodiment, in the undeployed state the base has a substantially linear shape. Thereby, the device may be delivered with a sleeve-like delivery device.

In a preferred embodiment, the base includes at least one flexible element and at least one rigid element, wherein the flexible element allows an expansion and/or compression along a longitudinal direction of the base.

In a preferred embodiment, the base comprises at least two rigid elements which are connected by a flexible element.

In a preferred embodiment, the base comprises alternating rigid and flexible elements. In a preferred embodiment, the rigid element is arranged movably within the base such that it is pushable towards an inner annular area. In a preferred embodiment, the at least one rigid element includes an interface, preferably an opening, for a connection to a cage.

In a preferred embodiment, the rigid element comprises a first pad, which extends along a plane of the annular shape and a second pad which is angled relative to the first surface. In particular, an angle between first and second plate is 30 to 150°, preferably 60 to 150, particularly preferred 75 to 1150. The second plate may provide a pushing surface for a manipulator.

The first plate may additionally comprise an anchor. The first plate may be connected to one or two or more flexible elements. The first and/or second pad may be sections of a bent plate.

Furthermore the rigid element may comprise an attachment element for the tissue, additionally or alternatively to the anchor. The attachment element may be a barb or a plurality of barbs. Preferably, each rigid element comprises an attachment element. Thereby, the base may include pre-attachment elements which allow a fixation prior to a final anchoring by the anchors.

In a preferred embodiment, the flexible element comprises a wire, wherein the wire has an undulating shape. Thereby, the base can be compressed and/or expanded along its longitudinal direction.

In a preferred embodiment, the wire comprises a first and second end and each end is connected to a rigid element. Preferably, the wire is connected to the first plate of the rigid element. Thereby, the flexible element between two rigid elements is formed in a simple manner.

In a preferred embodiment, the annuloplasty device comprises at least one commissural anchor for anchoring the base in an annulus. Preferably, the annuloplasty device comprises two commissural anchors for anchoring the base in an annulus. The annuloplasty device may additionally comprise one, two or more lateral anchors. The base and the cage may be attached to each other with at least one commissural anchor and/or at least one of the lateral lateral anchors. The lateral anchors may have an identical or similar structure and/or materials as the commissural anchor(s).

The commissural anchor(s) may be integral with the base or cage. In one embodiment the commissural anchor(s) are connectable to the base and/or cage. In one embodiment only a distal commissural anchor is connectable to the base and/or cage. A proximal commissural anchor may form a gate through which the base and/or cage extend. In another embodiment, at least one of the commissural anchors, preferably all, are integral with the base or the cage.

In a preferred embodiment, a first commissural anchor is disposed and at a distal end of the base. Preferably, the first commissural anchor is fixedly attached to the base at its distal end. Thereby, when a commissural anchor is placed first during implantation, the base may be attached in a simple fashion.

In a preferred embodiment, a second commissural anchor is disposed at an intermediary section along the longitudinal direction of the base.

In a preferred embodiment, the first and second commissural anchors are exteriorized independently from each other from a delivery device.

In a preferred embodiment, that the annuloplasty device comprises two commissural anchors wherein a distance in between the two commissural anchors is adjustable. Thereby, an inter-commissural distance of the valve annulus is adjustable according to the valve annulus and/or according to a desired shape of the native valve annulus.

In a preferred embodiment, that one or two or more commissural anchors are made from or comprise a shape memory alloy such that once a sleeve covering the commissural anchor is removed, the anchors are deployed and adapted to penetrate the native tissue of the annulus so to realize two commissural sutures. Thereby, the commissural anchors may be anchored to the native valve annulus. Particularly preferred, the commissural anchor(s) comprise one or two or more hooks which are made of a shape memory alloy. The hooks may be cut outs on an annularly outer surface of the commissural anchor.

In a preferred embodiment, the device comprises at least one sensor configured to detect regurgitation, in particular a sensor as suggested in WO 2016/174210.

In a preferred embodiment the annuloplasty device comprises an attachment interface for a prosthetic heart valve. The prosthetic heart valve may be attached to the annuloplasty ring and replace one or both native valve leaflets. Thereby, the annuloplasty device can be used as a landing platform for the prosthetic valve. The prosthetic valve may be percutaneously implantable. The interface may comprise struts, engagement elements and/or snapping elements.

The adjustment of the cage and/or base may be reversible. For example the inner wall may be made of a shape memory alloy. In another example the inner wall is pullable towards the outer wall, e.g. with a hook or by heating a shape memory material or by expanding a balloon in the native annulus opening. In particular the cage and/or base may be adapted to recover an initial position. Thereby, a center opening of the annular shape may be enlarged. Additionally the annuloplasty device could be base for a mitral valve without unnecessarily restricting the annulus.

Another aspect of the invention concerns a delivery system for an annuloplasty device. The delivery system comprises an outer sleeve, at least one commissural anchor, an inner sleeve, and an annuloplasty device. The annuloplasty device is disposed or disposable within the inner sleeve. The anchor is disposed between the inner and the outer sleeve. Thereby, the commissural anchors may be deployed before the annuloplasty device is released. This allows an adjustment of an independent placement of commissural anchor an annuloplasty device.

In a preferred embodiment, the annuloplasty device comprises a cage and a separate base wherein the cage and the base are disposed or disposable in the inner sleeve.

In a preferred embodiment, the base covers the cage at least partially. Thereby, during implantation the cage and the base may be inserted one after another or at the same time.

In a preferred embodiment, a first commissural anchor is disposed at a distal end of the delivery system. Thereby, the first commissural anchor may be deployed first.

In a preferred embodiment, a second commissural anchor is disposed at an intermediary section of the delivery system. Thereby, the annuloplasty ring may assume a first position before the second commissural anchor is fixed. This allows a flexible implantation procedure.

In a preferred embodiment, the delivery system comprises a spider. The spider is engageable to the annuloplasty device along the annular shape. The spider may have four legs, preferably like a “X”. The spider may extend across a center of the annuloplasty device, in particular across a native valve lumen. Thereby, a shaping and/or placement of the device could be facilitated. The spider might be adapted to remain implanted, e.g. in an atrial position. The spider is preferably made of or comprises a bioerodable material like magnesium or a magnesium alloy. The spider may comprise additionally or alternatively a shape memory material.

The legs of the spider may be collectively or individually pullable or pushable. Thereby, a shape of the annuloplasty device may be adjusted.

Another aspect of the invention concerns a delivery system for an annuloplasty device comprising a delivery tube and a second part adapted to be transported through the outer sleeve. The system comprises a position sensor for measuring a relative position of the second part in relation to the outer sleeve. Thereby, a current position of the second part may be detected.

The position sensor may be, but is not limited to, a linear encoder, in particular an optic, a magnetic, a capacitive, an inductive or an eddy current linear encoder. Alternatively the position sensor may be an accelerometer, in particular a MEMS accelerometer. The position sensor may include an electronic interface for a computing device. Alternatively or additionally the delivery system may comprise a memory for storing data collected by the position sensor.

In a preferred embodiment, the sensor is disposed at a distal port of the delivery system. Thereby, the position sensor may be conveniently read out.

In a preferred embodiment, the second part is at least one of: catheter, in particular a balloon catheter, a sleeve, in particular an inner or outer sleeve, an annuloplasty device.

In a preferred embodiment, the system comprises a display monitor for indicating a current relative position of the second part to a user.

Another aspect of the invention concerns a method for deploying an annuloplasty device comprising the steps of:
Deploying a first commissural anchor from a distal end a delivery device,
Securing the first commissural anchor to a native annulus
Bringing an intermediary section of the delivery device to a desired position opposing the first commissural anchor and deploying and securing a second commissural anchor to the native valve annulus.
Deploying a longitudinally extending base from the distal end of the delivery device, wherein the base has a basically annular shape or is brought into an annular shape upon release from a delivery device, and wherein the base is flexible along its longitudinal direction, and
Anchoring the base to the native valve annulus.

Thereby, an inter-commissural distance may be varied according to the natural valve annulus and a desired reshaping of the inter-commissural distance may be achieved.

The first commissural anchor is preferably secured to the anterior commissure and the second commissural anchor to the posterior commissure of the native valve.

Further, a first lateral and/or a second lateral anchor may be deployed. The first and second lateral anchor may define an anterior-posterior distance of the annulus. The first lateral anchor may be secured to the posterior annulus, preferably in a position on the annulus corresponding to a P2 scallop of the mitral valve. The second lateral anchor may be deployed to the anterior annulus, preferably in a position on the annulus corresponding to an A2 scallop of the mitral valve. Thereby both main directions and lengths, i.e. anterior-posterior distance and inter-commissural distance, are defined.

In a preferred embodiment, after deploying and the securing the second commissural anchor and preferably before anchoring the base, the method further includes the step:
Pulling and/or pushing the delivery device to adjust the annular shape of the base.

Thereby a distance between two valve leaflets may be varied according to the natural valve annulus and a desired reshaping.

In a preferred embodiment, before deploying and the securing the second commissural anchor the method further includes the step: Pulling and/or pushing the delivery device to adjust a distance in between the first and the second commissural anchor. Thereby, the inter-commissural distance may varied without varying the distance between the commissural anchors when they are in a delivery device

In a preferred embodiment, after deploying and the securing the second commissural anchor the method further includes the steps of deploying a longitudinally extending tubular cage having at least partially the basically annular shape of the base or bringing the cage at least partially into the annular shape of the base upon release from the distal end of a delivery device and preferably attaching the cage to the base.

In a preferred embodiment, the step of anchoring the base to the native valve annulus is conducted simultaneously to the step of attaching the cage to the base. Thereby, an implantation procedure is accelerated.

In a preferred embodiment, during the attaching step, anchors are deployed. The anchors include a pin with an elongate body part and a preferably sharp tip. The anchors are preferably attached to the base and/or cage. In a preferred embodiment, the cage is rigid along its longitudinal direction.

In a preferred embodiment, the method additionally includes the steps of introducing a manipulator, preferably a balloon, into the annuloplasty device and pushing a portion of an inner wall of the cage towards an inner annular area of the base. The inner wall is less rigid than an outer wall of the cage. Examples for a structure of the cage are shown in WO 2015/121075.

In a preferred embodiment, the method includes the step of deploying a first and/or second commissural anchor by moving an outer sleeve relatively to the respective commissural anchor, preferably by retracting the outer sleeve.

In a preferred embodiment, the method includes the step of deploying the base by moving an inner sleeve relatively to the base, preferably by retracting the inner sleeve.

Another aspect of the invention concerns a method for displaying an annuloplasty delivery system in an augmented reality environment performed by one or more processing devices comprising the steps of:
Receiving position data via an interface, wherein the position data includes distances of a delivery tube for an annuloplasty device relative to another part of a delivery system for an annuloplasty device
Combining the position data with a model of an annuloplasty device, which is stored in a memory of the processing device, wherein the model comprises data of an, preferably three dimensional, image of the part of a delivery system,
Calculating a current position of the part of a delivery system relative to the delivery tube
Deducing a preferably 3-dimensional shape of the part of a delivery system
Rendering an image, preferably a 3-dimensional image, corresponding to the delivery tube and the part of the delivery system, that is animated based on the shape of the delivery system
Showing the image, preferably the 3-dimensional image, indicating the current position and shape of the part of a delivery system to a user.

Thereby, an augmented reality with imaging fusion is provided, in which data from a model of the device and measured data are combined. The augmented reality is usable independently of an operation. For example, the position data could be saved in an electronic storage and the image is rendered and showed after the operation. In another example the position data could be generated without a position sensor. Thereby, an operation could be planned in advance of the actual procedure.

During or after an implantation procedure a physician may monitor the current state and positon of the part of the delivery system. This enables the physician either to observe previously non-observable items such as a sleeve or a balloon catheter made out of plastic. Alternatively or additionally the method reduces a reliance on traditional invasive observation methods such as fluoroscopy.

In a preferred embodiment, the method comprises the step of measuring position data with a position sensor, wherein the position sensor measures a relative position between an outer sleeve and at least one of: a catheter, a sleeve, an annuloplasty device and a balloon. The method may further comprise the step of sending the position data to an interface of a processing device, preferably sending a live feed to the processing device. Thereby, the current state may be monitored during an operation.

In a preferred embodiment, the part of the annuloplasty device is at least one of: an adjustable annuloplasty device, a base of an adjustable annuloplasty device, a cage of an adjustable annuloplasty device, a commissural anchor for an adjustable annuloplasty device, an outer sleeve, an inner sleeve, and a balloon catheter.

In a preferred embodiment the method further comprises the steps of recording a imaging data with an echo-fluoroscopy imaging device, wherein the imaging data is preferably 3 dimensional, and receiving the imaging data from the echo-fluoroscopy imaging device. Additionally or alternatively the echo-fluoroscopy imaging data may be generated beforehand and stored.

In a preferred embodiment the method further comprises the step of generating a virtual augmentation corresponding to the delivery tube and the part of the delivery system, which is animated based on the echo-fluoroscopy imaging. The step may include displaying an overlap between the echo-fluoroscopy imaging data and the animated image. The method may animate the whole delivery system or only parts. Thereby, position data and model are combined with fluoroscopy data. This allows training for operations, planning of operations, improving the operation itself and/or a post-operation audit.

In a preferred embodiment, the method comprises the step of comparing the echo-fluoroscopy data to the deduced shape of the part of the delivery system. Thereby, current state may be monitored more precisely. A user may be informed about a difference between the deduced shape and the detected shape. In particular, the difference may also be displayed.

In a preferred embodiment the method further comprises the step of rendering a sequence of images corresponding to the delivery tube and the part of the delivery system. Thereby, a stream may be provided.

In a preferred embodiment the method further comprises the step of receiving a sequence of position data, wherein the position data corresponds to a current position of the annuloplasty device. Thereby, a stream showing a movement of the device may be shown.

Another aspect of the invention concerns a computer-readable storage memory containing a computer program product for displaying a state of an annuloplasty delivery system by performing a method for displaying a state of an annuloplasty delivery.

Another aspect of the invention concerns one or more processing devices adapted to perform a method for displaying an annuloplasty delivery system.

Another aspect of the invention concerns an augmented reality system comprising a computer adapted to perform a method for displaying an annuloplasty delivery system and a delivery system for percutaneous delivery of an annuloplasty system.

Non-limiting embodiments of the invention are described, by way of example only, with respect to the accompanying drawings, in which:

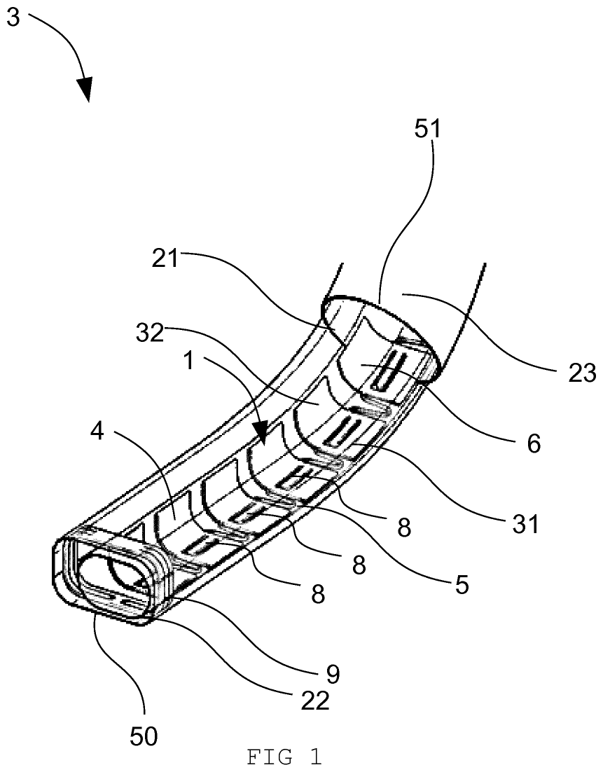

FIG. 1: is a schematic perspective view of a delivery system with an annuloplasty device during a first step of implantation of an annuloplasty device,

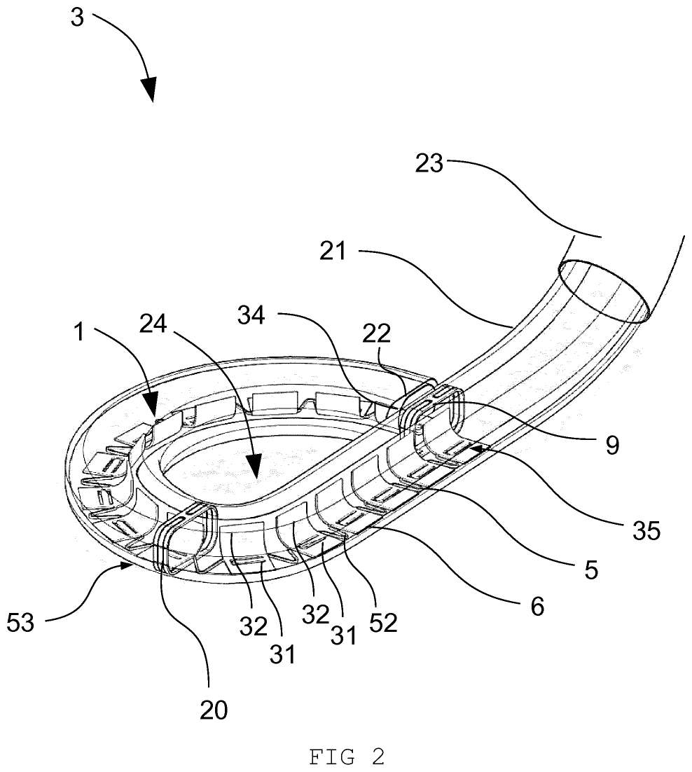

FIG. 2: is a first schematic perspective view of the delivery system with the annuloplasty device during a second step of implantation of an annuloplasty device,

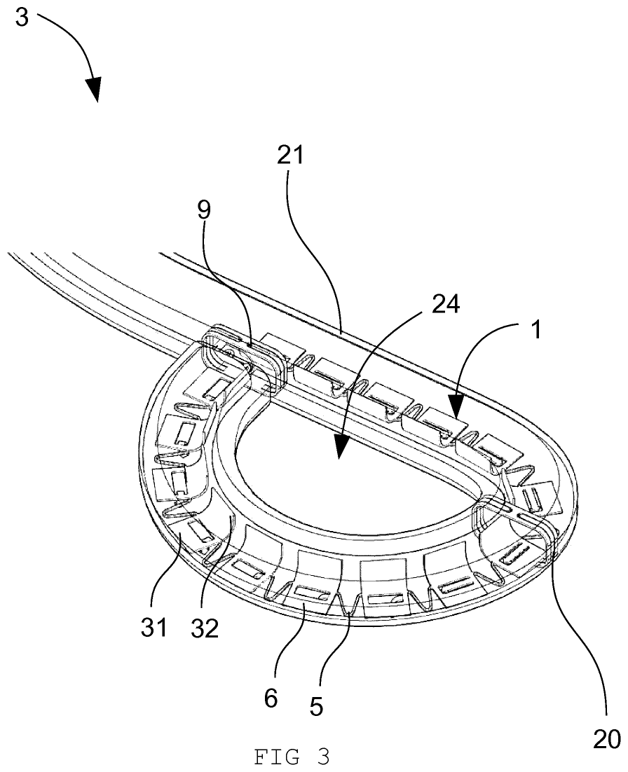

FIG. 3: is a second schematic perspective view of the delivery system with the annuloplasty device during the second step of implantation of an annuloplasty device,

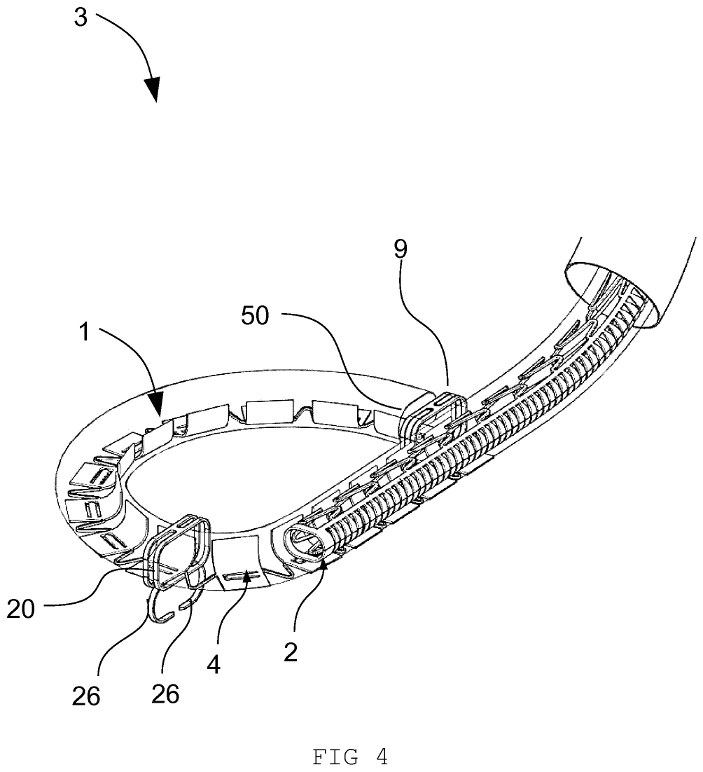

FIG. 4: is a first schematic perspective view of the delivery system with the annuloplasty device during a third step of implantation of an annuloplasty device,

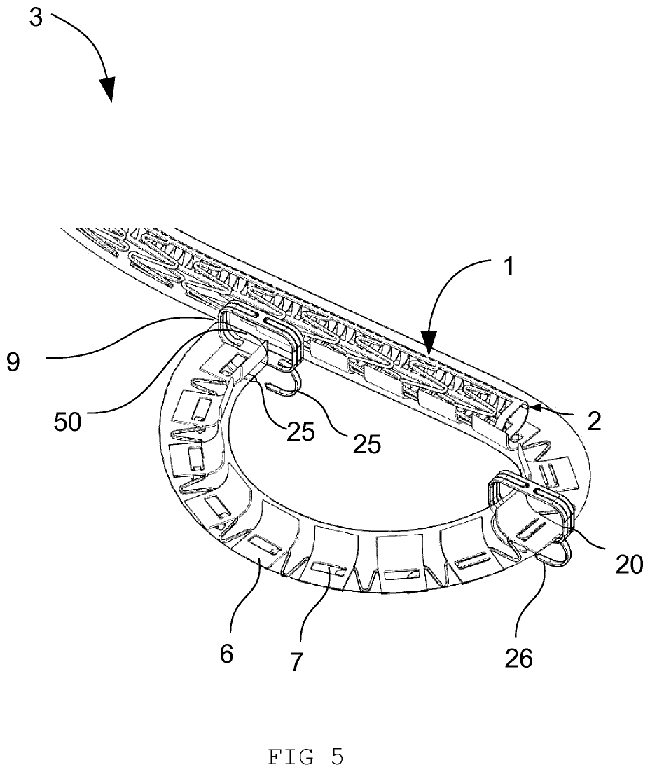

FIG. 5: is a second schematic perspective view of the delivery system with the annuloplasty device during the third step of implantation of an annuloplasty device,

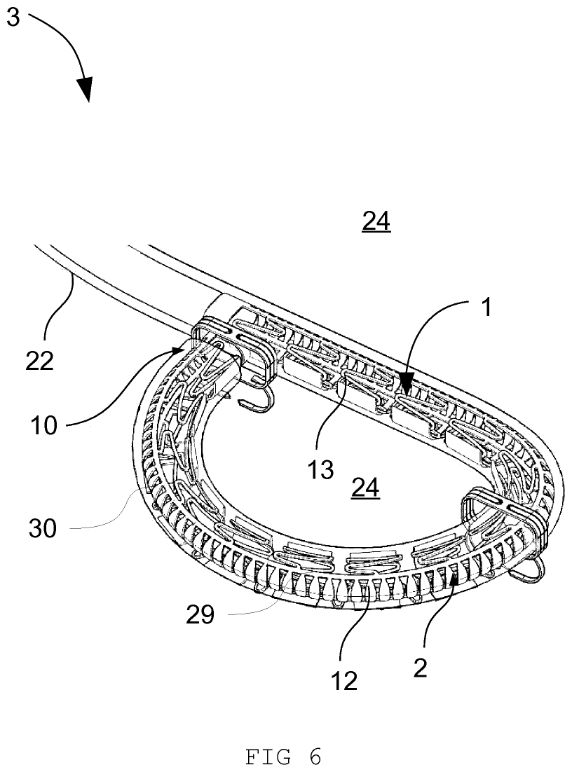

FIG. 6: is a first schematic perspective view of the delivery system with the annuloplasty device during a fourth step of implantation of an annuloplasty device,

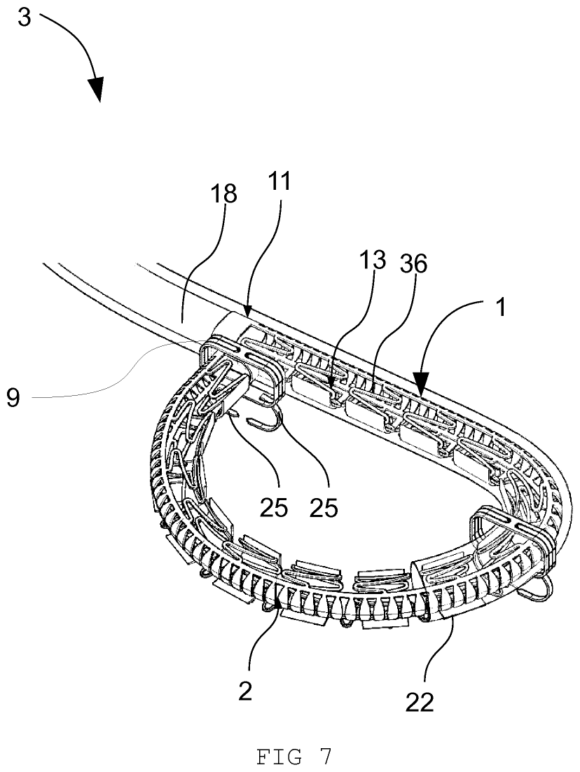

FIG. 7: is a second schematic perspective view of the delivery system with the annuloplasty device during a fifth step of implantation of an annuloplasty device,

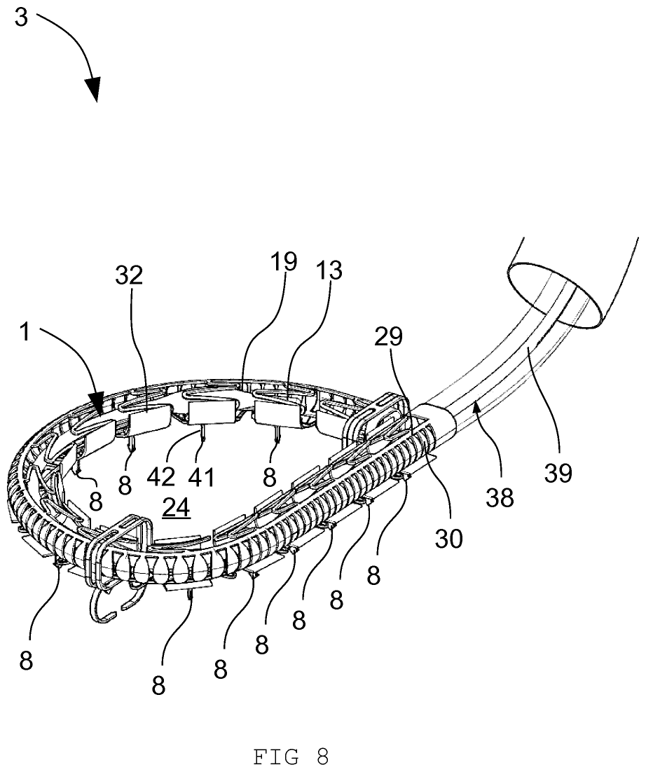

FIG. 8: is a first schematic perspective view of the delivery system with the annuloplasty device during a sixth step of implantation of an annuloplasty device,

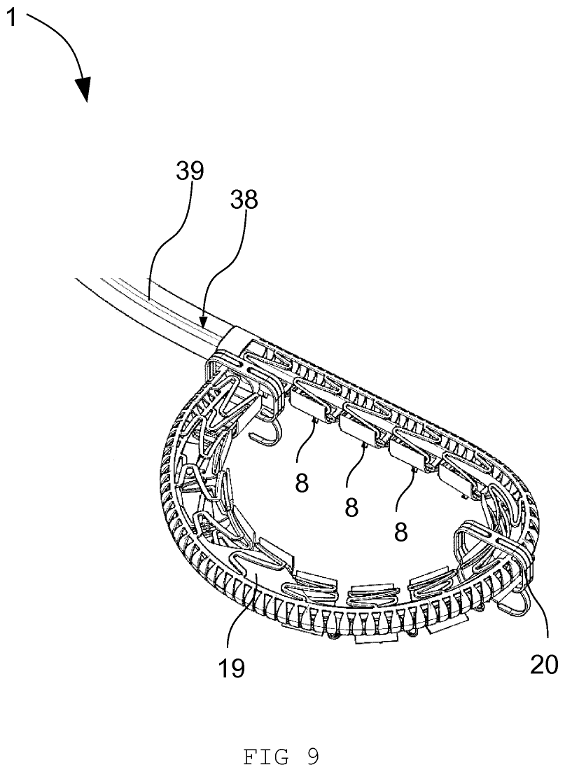

FIG. 9: is a second schematic perspective view of the delivery system with the annuloplasty device during the sixth step of implantation of an annuloplasty device,

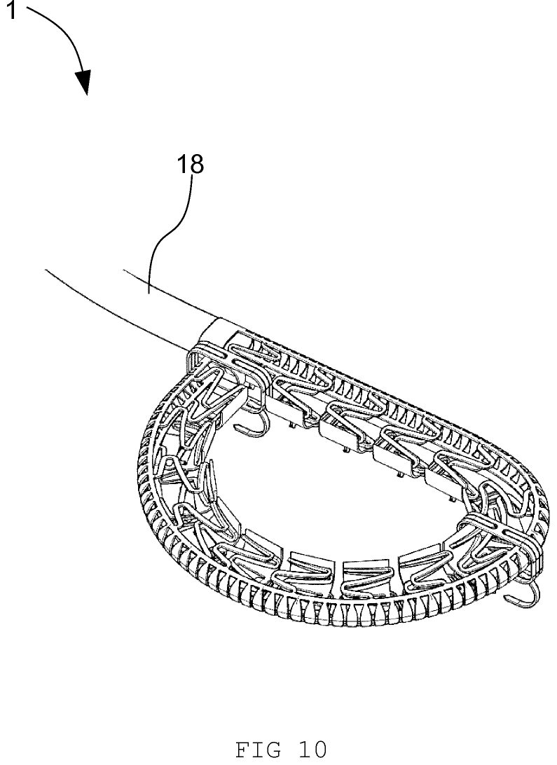

FIG. 10: is a schematic perspective view of the implanted annuloplasty device and

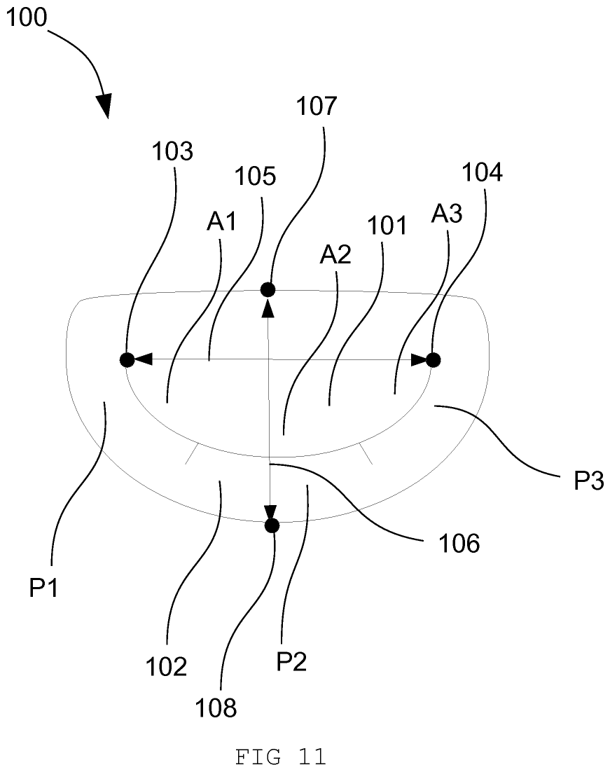

FIG. 11: is a schematic drawing of a mitral valve as seen from the left atrium.

FIG. 11 shows a schematic drawing of a mitral valve as seen from the left atrium. The mitral valve 100 comprises an anterior leaflet 101 and a posterior leaflet 102. Both leaflets 101, 102, extend into the left ventricle, where they co-apt. The leaflets 101, and 102, are biased towards the left ventricle and kept from prolapsing into the left atrium by chordae. Around the circumference of the mitral valve 100, the leaflets 101, 102 are integral with the valve annulus. The anterior and the posterior leaflet each comprise three scallops. The anterior scallops A1, A2, A3 and the posterior scallops P1, P2, P3 are positioned across each other such that in a healthy valve A1 and P1, A2 and P2, and, A3 and P3 coapt. Certain medical conditions may reduce a coaptation between the leaflets 101 and 102. As a result of the insufficient coaptation, there may be flow from the left ventricle to the left atrium. This type of blood flow is called regurgitation.

In order to inhibit the regurgitation, the valve annulus may be re-shaped. A position where the two leaflets end, 103 and 104, is called a commissure. A distance between the two commissures is an inter-commissural distance 105. Further, an anterior-posterior distance 106 is defined by the positions of two points 107 and 108. The point 107 corresponds to a position of the A2 scallop on the annulus. The point 108 corresponds to a position of the P2 scallop on the annulus.

To re-shape the annulus a physician can reduce the distance 106 between the anterior leaflet and the posterior leaflet to increase a coaptation area. Additionally the physician may also bring the commissures closer together, i.e. reduce the inter-commissural distance 105.

FIG. 1 shows a schematic drawing of a delivery system 3 with an annuloplasty device 1 and a delivery device during a first step of implantation of the annuloplasty device 1. The delivery device includes a delivery tube 23, an outer sleeve 21 and an inner sleeve 22. FIG. 1 shows the first step of implantation, in which the annuloplasty device 1 is released from a distal end 51 of the delivery tube 23. Within the delivery tube 23 the outer sleeve 21 and the inner sleeve 22 are disposed. The inner sleeve 22 is placed within the outer sleeve 21. The delivery tube 23 is relatively stiff compared to the inner and outer sleeves wherein inner and outer tubes are made of a softer material such that they may assume an annular shape. However, the delivery tube 23 is adapted for the transatrial approach. Thus the tube is flexible enough for the sharp turns in the aortic arch and through a left ventricle. In between the inner sleeve 22 and the outer sleeve 21, a first commissural anchor 9 is disposed at a distal end 50 of the outer tube 21. The first commissural anchor 9 is later placed in proximity to the anterior commissure 103 of the native valve and thus called anterior commissural anchor 9 in the following. Within the inner tube 22 a base 4 is held. The base 4 comprises of alternating rigid elements 6 and flexible elements 5.

Before inner and outer sleeve 21, 22 are released from the delivery tube 23, the delivery tube 23 is brought to a suitable location, i.e. a mitral valve annulus. The delivery system 3 is brought to the mitral valve annulus either through a transseptal or a trans-femoral or a trans-apical approach. The transseptal approach is preferred. Once a distal end 51 of the delivery tube 23 is in a suitable location, i.e. the mitral valve annulus, the release procedure of the annuloplasty device 1 is initiated by exteriorizing the inner and outer sleeves 21, 22 simultaneously as shown in FIG. 1.

The rigid element 6 includes a plate which comprises a lower pad 31 and an angled pad 32. In an implanted position, the lower pad 31 is anchored to tissue of a native valve annulus. The pads 31, 32 are made from the single plate which is bent in a middle part. The rigid elements 6 are interconnected by the flexible elements 5. As can be seen from FIG. 1, the lower pad 31 comprises a U-shaped cut out. The U-shaped cut out defines anchors 8.

FIGS. 2 and 3 show a first and a second schematic view of the delivery system 3 with the annuloplasty device 1 during a second step of implantation. As inner and outer sleeves 21, 22 are further pushed out of the delivery tube 23, the entire base 5 is released from the delivery tube 23. The base 5 is made from a shape memory alloy nitinol. The shape memory alloy is biased such that upon release the base 5 assumes a D-shape. Each flexible element 5 consists of a wire 52 which has an undulating shape. The wires 52 are made from a shape memory of material such that a selection of the wires expands. As a result, outer sleeve, inner sleeve and base assume a D-shape, once they are released from the stiffer delivery tube 23. The angled pad 32 faces towards the inner annular area 24. In the D-shape a distal end 34 of the base 4 and the anterior commissural anchor 9 are in contact which a proximal part 35 of the outer sleeve 21.

Further to the distally disposed commissural anchor 9, the delivery system comprises a second commissural anchor 20. The second commissural anchor 20 is located at an intermediary part 53 of the base is also disposed between inner and outer sleeve 21, 22. The second commissural anchor 20 is later placed in proximity to the posterior commissure 104 of the native valve and thus called posterior commissural anchor 20 in the following. Once the base 5 assumes the D-shape upon leaving delivery tube 23, the first and second commissural anchors are located at the “corners” of the D-shape.

During implantation the D-shape is aligned with the annulus of the mitral valve. The anterior commissural anchor 9 is arranged such that the anterior commissural anchor 9 is placed on the anterior commissure 103 of a native valve annulus. Then the outer sleeve 21 is retracted.

FIGS. 4 and 5 show a first and second schematic view of the delivery system 3 with the annuloplasty device 1 during a third step of implantation. FIGS. 4 and 5 show the delivery system 3 after the retraction of the outer sleeve 21. Both commissural anchors 9 and 20, are made of a shape memory alloy. The commissural anchors 9, 20 have a ring shape with cut outs. The cut outs extend a circumference of the commissural anchors 9 and 20 and define hooks 25, 26. As long as the commissural anchors 9 and 20 are held in the outer sleeve 21, these fingers are held within the annular shape of the commissural anchors 9 and 20. However, after retraction of the outer sleeve, the hooks 25, 26 bend out of the annular shape and fixate the commissural anchors 9, 20 in a tissue of the natural valve annulus. The retraction of the outer sleeve 21 is made in two steps. In a first step, under imaging guidance and after the anterior commissural anchor 9 is aligned with the anterior commissure 103 of the mitral valve (see FIG. 11), the anterior commissural anchor 9 is released from the distal end 50 of the outer sleeve 21. Then, a position of the posterior commissural anchor 20 is found by pulling or pushing the delivery system forwards or backwards until the posterior commissural anchor 20 is in a desired position. Thus, while the anterior commissural anchor 9 is in a fixed position, the position of the posterior commissural anchor 20 may still be freely defined. Thereby, an inter-commissural distance 105 may be set according to a desired geometry after the annuloplasty. Once a suitable position is found, the outer sleeve 21 is fully retracted. Thereby, the hooks 26 of the posterior commissural anchor 20 are also released.

Then in a next step, the anterior-posterior distance is adjusted for a first time. Base 4 is fixedly attached to the first commissural anchor 9. Base 4 is not fixedly attached to the posterior commissural anchor 20. The base extends through the ring shape posterior commissural anchor 20. Thus, the base may be pushed distally and proximally through the posterior commissural anchor 20 which acts as a gate. The interior posterior distance is then adjusted by pulling or pushing the inner sleeve creating a smaller or larger radius of a round part of the D-shape. Thereby, the size of the native valve annulus is adjusted. Thereafter a cage 2 is advanced through the inner sleeve 22.

FIG. 6 shows a schematic view of the delivery system 3 and the annuloplasty device 1 during a fourth step of implantation of the annuloplasty device 1. The cage 2 is advanced until its distal end 10 is distally beyond a distal end of the base. FIG. 6 shows the delivery system 3, once the cage 2 has been fully advanced in the inner sleeve 22. As can be seen from FIGS. 4 and 5, the base 4 has an L-shape, which covers a part of an inner surface of the inner sleeve 22.

The cage 2 has a tubular shape which defines an inner lumen. Thus, the cage 2 may be advanced through the lumen of the inner sleeve 22 while the base is in the lumen. The cage 2 comprises an outer wall 12 and an inner wall 13. Inner and outer wall 12, 13 are defined with regard to the annular shape of the annuloplasty device 1. Thus the inner wall 13 is a part of the cage 2 which faces an inner annular area 24 and the outside wall as a part of the wall which faces an outside area 15. The outer wall 12 is defined by two longitudinally extending struts 29, which are interconnected by scales 30. The longitudinally extending struts 29 define a length of the cage 2 and prohibit an extension and/or compression the base 4. The inner wall 13 comprises undulating struts which extend around the circumference of the tubular cage. These undulating struts may be expanded towards the inner annular area, as will be explained in greater details in FIGS. 8 to 10.

Like the base 4, the cage 2 is made from a nitinol and assumes upon exteriorization of the delivery tube 23 a D-shape, which facilitates advancing the cage 2.

FIG. 7 shows a schematic view of the delivery system of the annuloplasty device during a fifth step of implantation of the annuloplasty device 1. In FIG. 7 the inner sleeve 22 is partially retracted. The cage is advanced with a transmission line 18, which is attached to a proximal end 11 of the cage 2.

FIGS. 8 and 9 show a first and a second schematic view of the delivery system 3 with the annuloplasty device 1 during a sixth step of implantation of the annuloplasty device 1. Once cage 2 is fully advanced, the inner sleeve 22 may be withdrawn. In FIG. 7 the inner sleeve 22 is partially withdrawn. During withdrawal of inner sleeve 22 anchors 8 are deployed. The anchors include an elongate body part 42 and a sharp tip 41. When the anchors are deployed, they pivot outwardly into the tissue of the native valve.

During deployment the native valve annulus may be shaped. The anchors are deployed starting from a distal end. After first anchor 8 is placed a user pushes and/or pulls the delivery system to adjust a shape of the annulus. Then a second anchor is deployed. After the second anchor 8 is deployed the user may push or pull the delivery system again to align a position of the second anchor the corresponding tissue. The following anchors 8 are deployed in the same fashion until all anchors 8 are anchored in the native valve annulus and the native valve annulus is reshaped. During deployment, the anchors 8 are deployed along an inner edge of the annuloplasty device.

When the inner sleeve is retracted beyond the distal end of the base 4 and the cage 2, the distal end of the base 4 or the cage 2 or the anterior commissural anchor 9 interlocks with a proximal portion of the base or cage.

Once all anchors 8 are deployed, an initial shape of the annulus is set. This reshaping may be sufficient in order to inhibit regurgitation. However, if there is still residual regurgitation the annuloplasty device includes further adjustability. Furthermore, some patients, functional patients develop regurgitation after months or years of implantation of the annuloplasty device.

In both cases, the device is adjusted by advancing a balloon catheter 38 through the tubular transmission line 18 into the tubular shape of the cage 2. The balloon catheter 38 comprises a balloon 19 and a tube 39 through which the balloon 19 may be inflated. As can be seen from FIGS. 8 and 9, the outer wall 12 with its longitudinal struts 29 and scales 30 has a higher rigidity than the inner wall 12 with its undulating struts. Thus, if the balloon 19 of the balloon catheter 38 is expanded, the inner wall 12 is pushed towards the inner annular area 24.

The anchors 8 are deployed along an inner edge of the annuloplasty device 1. Upon expanding the balloon catheter 38, the anchors 8 and the annular tissue in which they are anchored are moved simultaneously inwardly.

Thereby, the angled pad 32 and with it the lower pad 31 and also anchors 8 are also pushed towards the inner annular area 24. As a result, the annuloplasty is adjusted by pushing only a section towards the inner annular area. The inner wall 13 is less rigid than the outer wall 12 along its entire length and thus the annuloplasty device 1 is adjusted around its entire annular shape is desired until there is no residual regurgitation.

FIG. 10 shows a schematic view of the implanted annuloplasty device. In FIG. 10 the balloon catheter 39 is removed from the annuloplasty device 1. FIG. 10 shows a final implanted shape of the implanted annuloplasty device 1. A transmission line 18 is attached to the cage. The transmission line 18 is a tube whose inner lumen is distally connected to a cage.

A user may insert a balloon catheter again into the transmission at a later time to readjust the annuloplasty device 1, if necessary.

A distal part of the delivery tube 23 comprises a linear encoder. The linear encoder measures a relative position between delivery tube 23 and outer sleeve 21. Also a second linear encoder measures a relative position between the inner sleeve 22 and the delivery tube 23. A third encoder measures a relative position between the transmission line 18 and the balloon catheter 39.

During implantation the measured relative positions are displayed to a surgeon. The relative positions indicate to the surgeon where the respective device is located, how much further they need to be advanced and in which shape the annuloplasty device currently is.

To aid the surgeon, their positions are visualised in a 3-D animation based on the shape memory properties of the annuloplasty device. The 3-D animation shows how the shape memory parts of the annuloplasty device assume their form without the need of further imaging technology. The 3-D animation shows the measured position in combination with 3-D model data with an imaging fusion.

Furthermore the 3-D data may be combined with imaging data from an echocardiography system. The echocardiography system provides fluoroscopic imaging data. The fluoroscopic imaging data is overlapped with the 3-D animation providing an augmented reality image or stream.

1-65. (canceled)

66. An adjustable percutaneous annuloplasty device comprising:
a tubular, longitudinally extending cage having a basically annular shape or being adapted to be brought into an annular shape upon release from a delivery device,
a separate, longitudinally extending base having at least a part of the basically annular shape of the cage or being adapted to be brought, at least partially, into the annular shape of the cage upon release from a delivery device,
wherein the cage is attachable to the base,
the base comprises at least one flexible element such that the annular shape of the base is adjustable, and
the flexible element allows at least one of an expansion and compression along a longitudinal direction of the base.

67. The adjustable annuloplasty device according to claim 66, wherein the cage and the base are independently insertable to an implantation site and adapted to be connected to each other at the implantation site.

68. The adjustable annuloplasty device according to claim 66, wherein the base additionally has at least two rigid elements which are connected by a flexible element.

69. The adjustable annuloplasty device according to claim 68, wherein the base comprises alternating rigid and flexible elements.

70. The adjustable annuloplasty device according to claim 68, wherein the at least one rigid element is movably arranged within the base such that the at least one rigid element is pushable towards an inner annular area.

71. The adjustable annuloplasty device according to claim 68, wherein the at least one rigid element includes an interface, for a connection to the cage.

72. The adjustable annuloplasty device according to claim 66, wherein the cage is adjustable,
the cage comprises an outer and an inner wall and at least one portion of the outer wall is more rigid than opposite portion(s) of the inner wall,
the inner wall is arranged nearer to an inside area defined by the annular shape than the outer wall such that the inner wall is adapted to be displaced inwardly, at least along less rigid portion(s) of the circumference, upon actuation by at least one actuation element while the outer wall remains basically constant.

73. The adjustable annuloplasty device according to claim 72, wherein the outer wall is more rigid along its entire length than the at least a part of the length of the inner wall.

74. The adjustable annuloplasty device according to claim 66, wherein the cage comprises anchors for at least one of engaging an annulus and for connecting the cage to the annular base.

75. The adjustable annuloplasty device according to claim 66, wherein the base comprises anchors for at least one of engaging an annulus and for connecting the cage to the annular base.

76. The adjustable annuloplasty device according to claim 74, wherein the anchors comprise a shape memory alloy.

77. The adjustable annuloplasty device according to claim 76, wherein the anchors comprise pins with a longitudinally extending body part and a tip.

78. The adjustable annuloplasty device according to claim 68, wherein the at least one rigid element includes an opening, and the anchors extend through or are adapted to extend through said opening.

79. The adjustable annuloplasty device according to claim 74, wherein the at least one rigid element includes an opening, and the anchors extend through or are adapted to extend through said opening.

80. The adjustable annuloplasty device according to claim 66, wherein the annuloplasty device additionally comprises a transmission line for adjusting at least one of the cage and annulus base.

81. An adjustable percutaneously annuloplasty device comprising:
a longitudinally extending base having a basically annular shape and a deployed state and an undeployed state,
wherein the base is flexible such that the annular shape of the base is adjustable at least one of during and after implantation, and
the base comprises a shape memory alloy such that in the deployed state, upon release from a delivery device, the base has an annular shape, and the base includes at least one flexible element, which allows at least one of an expansion and compression along a longitudinal direction of the base.

82. The adjustable annuloplasty device according to claim 81, wherein, the base includes at least one flexible element and at least two rigid elements, which are connected by the flexible element, and the flexible element allows at least one of an expansion and compression along a longitudinal direction of the base.

83. The adjustable annuloplasty device according to claim 82, wherein the base comprises alternating rigid and flexible elements.

84. The adjustable annuloplasty device according to claim 82, wherein the at least one rigid element is moveable arranged within the base such that the at least one rigid element is pushable towards an inner annular area.

85. The adjustable annuloplasty device according to claim 82, wherein the at least one rigid element includes an interface for a connection to a cage.

86. The adjustable annuloplasty device according to claim 82, wherein the rigid element comprises a first pad, which extends along a plane of the annular shape and a second pad which is angled relative to the first pad.

87. The adjustable annuloplasty device according to claim 86, wherein the flexible element comprises a wire and the wire comprises a first and second end and each end is a connected to a first surface of a rigid element.

88. The adjustable annuloplasty device according to claim 66, wherein the annuloplasty device comprises at least one commissural anchor for fixating the base to an annulus.

89. The adjustable annuloplasty device according to claim 88, wherein the commissural anchors comprises a shape memory alloy such that once a sleeve covering the commissural anchor is removed, the anchors are deployed and adapted to penetrate the native tissue of the annulus so to realize two commissural fixations.

90. A delivery system for percutaneously implanting an annuloplasty device comprising an outer sleeve, at least one commissural anchor, an inner sleeve, and an annuloplasty device,
wherein the annuloplasty device is arranged or arrangeable within the inner sleeve, and
the commissural anchor is disposed between the inner and the outer sleeve.

91. The delivery system according to claim 90, wherein the annuloplasty device comprises a cage and a base, and the cage and the base are arranged or arrangeable in the inner sleeve.

92. The delivery system according to claim 90, wherein the base at least partially covers an outer surface of the cage.

93. The delivery system according to claim 90, wherein a first commissural anchor is disposed at a distal end of the delivery system.

94. The delivery system according to claim 90, wherein the delivery system further comprises a delivery tube, and the outer sleeve is disposed within the delivery tube.

95. The delivery system according to claim 94, wherein the delivery tube is more rigid than at least one of the outer and the inner sleeve.

96. The delivery system according to claim 90, wherein a second commissural anchor is disposed at an intermediary section of the delivery system.

97. The delivery system for an annuloplasty device, comprising a delivery tube and a second part adapted to be transported throug h the outer sleeve,
wherein the system comprises a position sensor for measuring a relative position of the second part in relation to the outer sleeve.

98. The delivery system for an annuloplasty device according to claim 97, wherein the sensor is disposed at a distal port of the delivery system.

99. The delivery system for an annuloplasty device according to claim 97, wherein the second part is at least one of:
a catheter,
a sleeve,
an annuloplasty device, and
a balloon.

100. The delivery system for an annuloplasty device according to claim 97, wherein the system comprises a monitor for indicating a current position of the second part to a user.
